(19) United States
(12) Patent Application Publication
(10) Pub. No.: US 2019/0381046 A1
(43) Pub. Date: Dec. 19, 2019

(54) TOFACITINIB AND BACLOFEN COMPOSITIONS AND APPLICATIONS

(71) Applicant: ANDREW HANNA, CORNWALL (CA)

(72) Inventor: ANDREW HANNA, CORNWALL (CA)

(21) Appl. No.: 16/415,206

(22) Filed: May 17, 2019

Related U.S. Application Data
(60) Provisional application No. 62/684,993, filed on Jun. 14, 2018.

(51) Int. Cl. A61K 31/519; A61K 31/167; A61K 31/135; A61K 31/196; A61K 31/197; A61K 9/00; A61K 9/12; A61K 9/06

(52) U.S. Cl. CPC A61K 31/519; A61K 31/167; A61K 31/135; A61K 31/196; A61P 17/14; A61K 9/0014; A61K 9/122; A61K 9/06; A61K 31/197

(57) ABSTRACT

The human body is mediated by a large number of chemicals and chemical processes where imbalances can result in abnormal conditions affecting part or all of the human body. Artificial drugs addressing specific diseases by targeting specific chemical imbalance(s) and/or process(es). For example, tofactinib is a JAK-STAT signaling pathway inhibitor beneficial for treating autoimmune diseases whilst baclofen acts as an agonist of GABA receptors. However, these are each prescribed in oral form leading to reduced efficacy or absorption by other regions of the patient. Accordingly, the inventor has established alternate dosing formats such a topical creams allowing local direct application allowing specific targeted delivery to the appropriate region of the patient's body as well their use, in the instances of tofactinib and baclofen, for the treatment of other diseases or conditions.

### CROSS REFERENCE TO RELATED APPLICATIONS

This application claims priority from U.S. Provisional Patent application 62/684,993 filed Jun. 14, 2018 entitled “Tofacitinib and Baclofen Compositions and Applications”, the entire contents of which are incorporated herein by reference.

### FIELD OF THE INVENTION

This invention relates generally relates to medicaments and other agents and more specifically, the present invention relates to the delivery of medicaments or other agents such as β-(4-chlorophenyl)-γ-aminobutyric acid (β-(4-chlorophenyl)-GABA) and (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) within a topical lotion, cream, ointment gel, shampoo, or foam.

### BACKGROUND OF THE INVENTION

The human body is an extremely complex interlinked system mediated by a large number of chemicals and chemical processes. Accordingly, any imbalance of any one or more of these0 chemicals or chemical processes can result in an abnormal condition that affects part or all of the human body. Typically referred to as a “disease” or a “condition” in order to differentiate imbalances caused by an external force, an “‘injury”, then many diseases arise from an external infection or pathogen whilst others arise from biological degradations triggered from one or more factors of which age, genetics, and weight are the most common. A disease typically consists of a disorder of a structure or function. Over history a wide range of medicines have been used to address specific diseases although with the advancements in the 19^{th} and 20^{th} centuries in respect of diagnostic techniques etc. the number of identified diseases, their causes, and their medicinal treatments has increased substantially. With the concurrent developments in chemistry a wide range of artificial drugs have been added to the physician's armoury to address specific diseases by targeting the specific chemical imbalance(s) and/or chemical process(es).

Amongst these multitude of diseases are alopecia areata and vitiligo whilst amongst the multitude of conditions is pain.

Alopecia areata, also known as spot baldness, is a condition in which hair is lost from some or all areas of the body. Often it results in a few bald spots on the scalp although in extreme all the hair on the scalp or all body hair is lost and the loss permanent. Due to the nature of the visible bald spots in the sufferer's head this can lead to significant psychological stress, self-consciousness etc. Sufferers are generally otherwise healthy and the initial symptoms of alopecia areata are small bald patches where the underlying skin is unscarred and looks superficially normal. Alopecia areata most often affects the scalp and beard but may occur on any part of the body with hair. Different areas of the skin may exhibit hair loss and regrowth at the same time. The disease may also go into remission for a time or may be permanent. The area of hair loss may tingle or be painful and the hair tends to fall out over a short period of time, with the loss commonly occurring more on one side of the scalp than the other. Additionally, hair will tend to pull out more easily along the edge of the patch where the follicles are already being attacked by the body's immune system than away from the patch where they are still healthy.

In cases of severe hair loss, limited success has been achieved by using the corticosteroids clobetasol or fluocinonide, corticosteroid injections, or corticosteroid cream. Steroid injections are commonly used in sites where the areas of hair loss on the head are small or especially where eyebrow hair has been lost although their efficacy is uncertain. Some other medications that have been used include minoxidil, mometasone (steroid cream), irritants (anthralin or topical coal tar), and topical immunotherapy ciclosporin, sometimes in different combinations. Topical corticosteroids frequently fail to enter the skin deeply enough to affect the hair bulbs, which are the treatment target, and small lesions typically also regrow spontaneously. Oral corticosteroids may decrease the hair loss, but only for the period during which they are taken, and these drugs can cause serious side effects. No one treatment has to date been shown to be effective in all cases, and some individuals may show no response to any treatment.

Tofacitinib (tofacitinib citrate) may be effective at treating alopecia areata. Accordingly, it would be beneficial to provide patients with a topical cream of tofacitinib to apply to affected areas rather than employing it within an oral medication as currently taken by patients for rheumatoid arthritis.

Vitiligo is a long-term skin condition characterized by patches of the skin losing their pigment. The patches of skin affected become white and usually have sharp margins. The hair from the skin may also become white. Inside the mouth and nose may also be involved. Typically, both sides of the body are affected. Often the patches begin on areas of skin that are exposed to the sun. It is more noticeable in people with dark skin. Vitiligo may result in psychological stress and those affected may be stigmatized.

There is no cure for vitiligo, but several treatment options are available. The best evidence to date is for applied steroids and the combination of ultraviolet light in combination with a cream. At present topical preparations of immune suppressing medications including glucocorticoids (such as 0.05% clobetasol or 0.10% betamethasone) and calcineurin inhibitors (such as tacrolimus or pimecrolimus) are considered to be first-line vitiligo treatments.

However, tofactinib is an inhibitor of JAK-STAT signaling pathways which transmit extra-cellular information into the nucleus. It has been shown to be beneficial in treating other autoimmune diseases such as alopecia areata and accordingly as vitiligo is also believed to be caused by the immune system the inventor has established a topical cream of tofacitinib to apply to affected areas rather than employing it within an oral medication as currently taken by patients for rheumatoid arthritis.

Pain is the most common reason for a physician consultation in most developed countries and is a distressing feeling often caused by intense or damaging stimuli. The International Association for the Study of Pain's widely used definition defines pain as “an unpleasant sensory and emotional experience associated with actual or potential tissue damage, or described in terms of such damage”, however, due to it being a complex, subjective phenomenon, defining pain has been a challenge. In medical diagnosis, pain is regarded as a symptom of an underlying condition.

Acute pain is usually managed with medications such as analgesics and anesthetics. Examples of these include, but are not limited to, NSAIDs (non-steroidal anti-inflammatory drugs), including ibuprofen (Advil, Motrin), naproxen, and aspirin; acetaminophen (Tylenol); antidepressants, which can improve sleep and alleviate pain; anti-seizure medications, which can be effective in treating pain related to nerve damage or injury; and steroids, like dexamethasone and prednisone, to alleviate inflammation and pain.

Baclofen has been used to address spasticity which occurs in disorders of the central nervous system affecting the upper neurons by acting as an agonist at GABA receptors, specifically the GABA_{B} receptors, which are inhibitory. However, it is also postulated to block mono-and-polysynaptic reflexes by acting as an inhibitory neurotransmitter blocking the release of excitary transmitters. Accordingly, the inventor has established that baclofen may form the basis of a pain medication. However, it would be beneficial to provide a delivery mechanism of a topical cream to apply directly to the region(s) experiencing pain rather than employing the current tablet-based delivery mechanism.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations in the prior art relating to medicaments and other agents and more specifically, the present invention relates to the delivery of medicaments or other agents such as β-(4-chlorophenyl)-γ-aminobutyric acid (β-(4-chlorophenyl)-GABA) and (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) within a topical lotion, cream, ointment gel, shampoo, or foam.

In accordance with an embodiment of the invention there is provided a pharmaceutical composition comprising a janus kinase inhibitor and a topical carrier for the janus kinase inhibitor.

In accordance with an embodiment of the invention there is provided a pharmaceutical composition comprising (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) (tofacitinib) and a topical carrier for the tofacitinib.

In accordance with an embodiment of the invention there is provided a pharmaceutical composition comprising a GABA receptor activator inhibitor, and a topical carrier for the GABA receptor activator.

In accordance with an embodiment of the invention there is provided a pharmaceutical composition comprising β-(4-chlorophenyl)-γ-aminobutyric acid (β-(4-chlorophenyl)-GABA) (baclofen) and a topical carrier for the baclofen.

Other aspects and features of the present invention will become apparent to those ordinarily skilled in the art upon review of the following description of specific embodiments of the invention in conjunction with the accompanying figures.

### DETAILED DESCRIPTION

The present invention is directed to medicaments and other agents and more specifically, the present invention relates to the delivery of medicaments or other agents such as β-(4-chlorophenyl)-γ-aminobutyric acid (β-(4-chlorophenyl)-GABA) and (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) within a topical lotion, cream, ointment gel, shampoo, or foam.

The ensuing description provides exemplary embodiment(s) only, and is not intended to limit the scope, applicability or configuration of the disclosure. Rather, the ensuing description of the exemplary embodiment(s) will provide those skilled in the art with an enabling description for implementing an exemplary embodiment. It being understood that various changes may be made in the function and arrangement of elements without departing from the spirit and scope as set forth in the appended claims.

“Tofacitinib” or “tofacitinib citrate” as used herein and throughout the disclosure refers to the pharmaceutical compound (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) of chemical formula C₁₆H₂₀N₆O.C₆H₈O₇. Tofacitinib is a drug of the janus kinase (JAK) inhibitor class.

“Baclofen” as used herein and throughout the disclosure refers to the pharmaceutical compound β-(4-chlorophenyl)-γ-aminobutyric acid β-(4-chlorophenyl)-GABA) of chemical formula C₁₀H₁₂ClNO₂ and is a derivative of the neurotransmitter γ-aminobutyric acid (GABA).

The terms “drug” or “active agent” are used interchangeably herein and throughout the disclosure to describe a pharmacologically active substance that is present in the composition in a quantity that is sufficient to elicit an intended pharmacological response.

A “pharmaceutical composition” as used herein and throughout the disclosure refers to compositions comprising one or more drugs together with one or more pharmaceutically acceptable excipients as required to prepare a dosage form for the effective delivery of the active agent. Such pharmaceutical compositions can be in the form of solutions, ointments, creams, gels, lotions, suspensions, sprays, foams, microspheres, microemulsions, nanoemulsions, nanoparticles, nanosuspensions, dermal sticks, roll-ons, pumps, patches, tapes, and the like. Certain pharmaceutical compositions of the present invention may be in the form of “permeation enhanced” compositions, or “penetration enhanced” compositions, or “depot” compositions.

A “topical composition” or “topical” composition as used herein and throughout the disclosure refers to a composition that is applied onto the skin surface. Such a “topical” composition may act “locally” or “transdermally”. A “transdermal” composition refers to a composition that can be applied to the body surface, which then may permeate through the stratum corneum or scalp to form a reservoir just beneath the skin surface or may be absorbed systemically to provide desirable drug levels in the circulation.

“Skin depot” or “depot” as used herein and throughout the disclosure refers to a pharmaceutical composition that provides storage of drug within the skin and releases the contained drug to surrounding tissue over a prolonged period of time, and/or delayed to commence after a period of time.

The “scalp” as used herein and throughout the disclosure refers the skin covering the head and is bordered by the face anteriorly and the neck to the sides and posteriorly.

“Foamil” as used herein and throughout this disclosure refers to a foam which supports the compounding of active ingredient(s) without requiring heating and filtration steps as marketed those by Medisca under the trademark Foamil. Its composition makes it suitable for compounding other active ingredients that are appropriate for use in hair loss applications. The foam has a typical pH value above 3.5 and an alcohol concentration typically below 10% allowing it to be mild and non-irritating to the scalp, thus making it ideal for compounding leave-on treatments to prolong the life cycle of hair.

“VersaPro” as used herein and throughout this disclosure refers to a cream which supports the compounding of active ingredients as marketed by Medisca under the trademark VersaPro. The formulation is an oil-in-water formulation which exhibits strong drug compatibility and permeation, as well as non-comedogenic and hypoallergenic properties, rendering it beneficial for both pharmaceutical and cosmetic purposes. The product is non-greasy, non-irritant, and paraben-free. The VersaPro products include a cream base, lotion base, hormone replacement therapy base, and a transdermal pain base where the bases support lipophilic and hydrophilic pharmaceuticals.

“Dimethyl sulfoxide” (DMSO) as used herein and throughout this disclosure refers to an organosulfur compound with the formula (CH₃)₂SO. This colorless liquid is an important polar aprotic solvent that dissolves both polar and nonpolar compounds and is miscible in a wide range of organic solvents as well as water.

A “formulation” as used herein and throughout the disclosure refers to refers to any mixture of compositions used to make either a lotion according to embodiments of the invention or tablets according to embodiments of the invention.

An “organic solvent” and “organic solvent residue” as used herein and throughout the disclosure refers to an organic substance that dissolves a solute (a chemically distinct material), resulting in a solution. Such organic solvents may include, but not be limited to, an alcohol, isopropyl alcohol, ethanol, methanol, methylene chloride, acetone, and the like.

A “water soluble polymer” as used herein and throughout the disclosure refers to a polymeric composition, soluble in an aqueous solution.

“Polyvinylpyrrolidone” or “PVP” refers to any of the polymers of vinylpyrrolidone, or derivatives thereof. While PVP is typically made via a free radical polymerization process, any soluble grade polymer of vinylpyrrolidone may be employed within embodiments of the invention where appropriate. Typically, linear PVP polymers are water soluble and cross-linked PVP polymers are not water soluble.

A “saccharide” as used herein and throughout the disclosure refers to any monosaccharide or polysaccharide, or derivative thereof, from any natural or synthetic sources. A saccharide may include, but not be limited to, mannitol, lactose, glucose, sucrose, xylitol, maltose, maltitol, sorbitol, oligosaccharides, and other similar saccharides.

A “pharmaceutically active ingredient” as used herein and throughout the disclosure refers to any medicament, nutritional, palliative, drug or pharmaceutical added to a tablet or lotion as appropriate to the embodiment of the invention.

A “perfume” as used herein and throughout the disclosure refers to any composition that contributes to the odor or taste, or masks an unpleasant smell, of a formulation.

A “colorant” as used herein and throughout the disclosure refers to any composition that adds color to a formulation.

“Granulating” as used herein and throughout the disclosure refers to the process of blending and mixing a formulation.

“Compressing” as used herein and throughout the disclosure refers to the process of applying compressive force to a formulation, as within a die, to form a tablet.

“Humidifying” and “humidification” as used herein and throughout the disclosure refer to the process of adding moisture to a tablet, as reacting the tablet with a relatively humid (water saturated) environment. The term “relative humidity” is used in its common context and refers to the percentage of water saturation in a gas.

“Drying” and “dried” as used herein and throughout the disclosure refer to a process which decreases the water content of a composition, as the drying of a humidified tablet. A “dried tablet” as used herein and throughout the disclosure refers to a tablet that has been treated in any manner to decrease the amount of water in the formulation, as when a tablet is dried after its initial granulation and compression into a tablet form.

“Filler” as used herein and throughout the disclosure refers to any inert material or composition added to a formulation to add bulk to a formulation.

“Press molding” as used herein and throughout the disclosure refers to any apparatus which places compressive force on a formulation to compress and shape the composition, as with the compression of a wet or dry formulation to create a tablet.

“Physiologically acceptable” as used herein and throughout the disclosure refers to any combination of materials or compositions that are not harmful, i.e., non-toxic, to cells and tissues under physiologic (in vivo) conditions.

“Micronization” as used herein and throughout the disclosure refers to the process of reducing the average diameter of a solid material's particles. Traditional techniques for micronization focus on mechanical means, such as milling and grinding. Modern techniques make use of the properties of supercritical fluids and manipulate the principles of solubility. The term micronization usually refers to the reduction of average particle diameters to the micrometer range but can also describe further reduction to the nanometer scale. Common applications include the production of active chemical ingredients, foodstuff ingredients, and pharmaceuticals. These chemicals need to be micronized to increase efficacy.

A: Topical Tofacinib Composition

A1: Background

Alopecia areata is thought to be a systemic autoimmune disorder in which the body attacks its own anagen hair follicles and suppresses or stops hair growth. For example, T cell lymphocytes cluster around affected follicles, causing inflammation and subsequent hair loss. It is not contagious and tends to occur more frequently in people who have affected family members, suggesting heredity may be a factor. Alopecia areata shares genetic risk factors with other autoimmune diseases, including rheumatoid arthritis, type 1 diabetes, and celiac disease and in some instances nay be the only manifestation of celiac disease.

In 2010, a genome-wide association study identified 129 single nucleotide polymorphisms that were associated with alopecia areata. The genes that were identified include those involved in controlling the activation and proliferation of regulatory T cells, cytotoxic T lymphocyte-associated antigen 4, interleukin-2, interleukin-2 receptor A, and Eos (also known as Ikaros family zinc finger 4), as well as the human leukocyte antigen. The study also identified two genes, PRDX5 and STX17, that are expressed in the hair follicle.

At present with prior art treatments the objective assessment of treatment efficacy is very difficult and spontaneous remission is unpredictable, but if the affected area is patched, the hair may regrow spontaneously in many cases. None of the existing therapeutic options are curative or preventive. To date these prior art therapies have employed, for severe hair loss, the corticosteroids clobetasol or fluocinonide, corticosteroid injections, or a topical corticosteroid cream. The topical corticosteroid cream appears to be less effective and takes longer for results to be evident.

Steroid injections are commonly used in sites where the areas of hair loss on the head are small or especially where eyebrow hair has been lost. However, at present their efficacy is uncertain and such injections are not pleasant for the patient but their desire to regrow their hair and remove the psychological and social aspects of the disease significant. Other medications that have been used are minoxidil, mometasone ointment (a corticosteroid steroid cream), irritants such as anthralin or topical coal tar, and the topical immunosuppressant ciclosporin, sometimes in different combinations. Topical corticosteroids frequently fail to enter the skin deeply enough to affect the hair bulbs, which are the treatment target, and small lesions typically also regrow spontaneously. Oral corticosteroids may decrease the hair loss, but only for the period during which they are taken, and these drugs can cause serious side effects. No one prior art treatment is effective in all cases, and some individuals may show no response to any prior art treatment.

Many medications are being studied, including abatacept, MEXIS/M6S, triamcinolone, secukinumab, tralonkinumab, apremilast, botulinum toxin, INCB018424, bimatoprost, clobetasol, AS101, autologous platelet-rich plasma, topical minoxidil, and nitric oxide gel. Some of these medications are approved for other diseases, others are not available outside of specific studies.

In contrast, tofacitinib (tofacitinib citrate) [(3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1)], chemical formula C₁₆H₂₀N₆O.C₆H₈O₇, is a drug of the janus kinase (JAK) inhibitor class and is approved for the treatment of rheumatoid arthritis (RA) in the United States and other countries. It has demonstrated effectiveness in the treatment of psoriasis, is being studied for treatment of inflammatory bowel disease, and other immunological diseases, as well as for the prevention of organ transplant rejection.

At present Tofacitinib is available as Xeljanz which is typically taken at an immediate release form at 5 mg tofacitinib (equivalent to 8 mg tofacitinib citrate) orally two times a day or in extended release form at 11 mg tofacitinib (equivalent to 17.8 mg tofacitinib citrate) orally once day. Each 5 mg tablet of Xeljanz contains the appropriate amount of tofacitinib as a citrate salt and the following inactive ingredients: microcrystalline cellulose, lactose monohydrate, croscarmellose sodium, magnesium stearate, HPMC 2910/Hypromellose 6 cP, titanium dioxide, macrogol/PEG3350, and triacetin. Each 11 mg tablet of Xeljanz contains the appropriate amount of tofacitinib as a citrate salt and the following inactive ingredients: sorbitol, hydroxyethyl cellulose, copovidone, magnesium stearate, cellulose acetate, hydroxypropyl cellulose, HPMC 2910/Hypromellose, titanium dioxide, triacetin, and red iron oxide.

As tofacitinib is an inhibitor of the enzyme janus kinase 1 (JAK1) and janus kinase 3 (JAK 3), it interferes with the JAK-STAT signaling pathway, which transmits extracellular information into the cell nucleus, influencing DNA transcription. As janus kinases are both type 1 and type II cytokine receptors they can signal everything from apoptosis through to inflammation. Additionally, as they act as cytokine receptors, lack of JAK function can lead to inflammatory diseases (most notably skin diseases).

Accordingly, due to this ability to treat other autoimmune diseases then the diseases alopecia areata and vitiligo which are autoimmune and believed to be caused by the immune system respectively are diseases that tofacitinib can act as a medication for. Accordingly, the inventor has established a topical tofacitinib cream of tofacitinib to apply to affected areas rather than employing it within an oral medication as currently taken by patients for rheumatoid arthritis.

B2: Formulation Example

Accordingly, the inventor has established a topical tofacitinib cream for application by the user, for example a 2% tofacitinib cream (3.2% tofacitinib cream), as defined by Table 1. The selection of the base, in this instance Medisca Foamil™ depends upon the desired viscosity of the topical cream. Within other embodiments of the invention a topical cream may include a topical foam.

TABLE 1
2% Tofacitinib Cream

| Ingredient | Quantity | Unit |
|---|---|---|
| Tofacitinib Citrate | 6.186 | g |
| DMSO | 3.0 | mL |
| Medisca Foamil | 15.0 | mL |

TABLE 2
Suggested Tofacitinib Preparation for 100 g Batch

| Ingredient | Qty. | Unit |
|---|---|---|
| Tofacitinib Citrate | 3.200 | units |
| DMSO | 5.0 | mL |
| Medisca VersaPro ™ Lotion Base | 91.86 | g |
| (or VersaPro ™ Cream Base) | | |

Exemplary Tofacitinib Cream Preparation Process

Process 1: Powder to Liquid Preparation

An exemplary process for the preparation of a homogenous liquid-like dispersion of the tofacitinib citrate according to an embodiment of the invention comprises the following steps:
Step 1A: Triturate the tofacitinib citrate to form a fine, homogenous powder.
Step 1B: Levigate the fine homogeneous powder from step 1A with the ethoxy diglycol.
Step 1C: Sieve using 40-50 sieve mesh.

Process 2: Powder-Liquid to Base Incorporation

An exemplary process for the preparation of the medium from the homogenous liquid-like dispersion to a homogenous liquid and powder formulation according to an embodiment of the invention comprises the following steps:
Step 2A: Incrementally add the homogeneous liquid-like dispersion from step 1B to the VersaPro™ Lotion Base (or VersaPro™ Cream Base) whilst continuously mixing, using a high-shear mixing technique (for example using a Mazerustar Revolutionary Planetary Mixer at 2000 rpm for 30 seconds.

Process 3: Product Transfer

An exemplary process for the preparation of a 2% stock liquid solution according to an embodiment of the invention comprises the following steps:
Step 3A: Transfer final product from Process 2 to designated dispensing container(s).
If a foam base is employed within rather than a cream base then a foam dispensing pump should be employed for dispensing.

Process 4: Product Labelling

Once the cream-based (or foam-based) tofacitinib citrate is packaged in the dispensing container(s) then the dispensing container(s) should be labelled in compliance with the product labelling, drug regulations, etc. in the jurisdiction that the topical finasteride foam will be sold. For example, the product labelling may include for example the following elements as per United States pharmacopeia (USP Chapter 795).
Packaging Tightly closed, light-resistant ointment tube/jar/etc. Administer with metered dose measuring device.
Estimated Beyond-Use Date 30 days.
Label 1 Use as prescribed. Do not exceed prescribed dose.
Label 2 Keep out of the reach of children.
Label 3 Cap tightly after use.
Label 4 For external use only.
Label 5 Keep in a dry place.
Storage Label 1 Room temperature storage (20° C.-23° C.).
Storage Label 2 Protect from light.
Storage Label 3 Shake well before use.

Add auxiliary labels specific to the API to the dispensing container as deemed necessary including but not limited to:
Add auxiliary labels specific to the API to the dispensing container as deemed necessary including but not limited to:
Contact the pharmacist in the event of adverse reactions.
Consult health care practitioner if any prescription or over-the-counter medications are currently being used or are prescribed for future use.
The quantity of the API administered is directly dependent on the quantity of the product applied.

B: Topical Baclofen Composition

B1: Background

Pain is a distressing feeling often caused by intense or damaging stimuli. Acute pain is usually managed with medications such as analgesics and anesthetics. Examples of these include, but are not limited to, NSAIDs (non-steroidal anti-inflammatory drugs), including ibuprofen (Advil, Motrin), naproxen, and aspirin; acetaminophen (Tylenol); antidepressants, which can improve sleep and alleviate pain; anti-seizure medications, which can be effective in treating pain related to nerve damage or injury; and steroids, like dexamethasone and prednisone, to alleviate inflammation and pain.

Baclofen has been used to address spasticity which occurs in disorders of the central nervous system affecting the upper neurons by acting as an agonist at GABA receptors, specifically the GABA_{B} receptors, which are inhibitory. However, it is also postulated to block mono-and-polysynaptic reflexes by acting as an inhibitory neurotransmitter blocking the release of excitary transmitters. Accordingly, the inventor has established that baclofen may form the basis of a pain medication. However, it would be beneficial to provide a delivery mechanism of a topical cream to apply directly to the region(s) experiencing pain rather than employing the current tablet-based delivery mechanism.

Baclofen [β-(4-chlorophenyl)-γ-aminobutyric acid (β-(4-chlorophenyl)-GABA)], chemical formula C₁₀H₁₂ClNO₂, is a derivative of the neurotransmitter γ-aminobutyric acid (GABA). It is believed to work by activating (or agonizing) GABA receptors, specifically the GABA_{B} receptors. It is currently marketed under the tradename Lioresal for the treatment of spasticity and is typically started at a dosage of 15 mg daily and gradually increased wherein satisfactory control of patient symptoms is usually obtained with doses of up to 60 mg daily for spasticity, but a careful adjustment is often necessary to meet the requirements of each individual patient. The dose may be increased slowly if required, but a maximum daily dose of more than 100 mg is not advised unless the patient is in hospital under careful medical supervision. Each 10 mg or 20 mg tablet of Lioresal contains the appropriate amount of baclofen and the following inactive ingredients: silica aerogel (colloidal silicon dioxide), microcrystalline cellulose, magnesium stearate, povidone, and wheat starch (potato starch).

However, as baclofen is also postulated to block mono-and-polysynaptic reflexes by acting as an inhibitory neurotransmitter blocking the release of excitary transmitters, the inventor has established that baclofen may form the basis of a pain medication. However, it would be beneficial to provide a delivery mechanism of a topical cream to apply directly to the region(s) experiencing pain rather than employing the current tablet-based delivery mechanism.

Exemplary Baclofen Cream Preparation Process

Accordingly, the inventor has established a topical baclofen cream for application by the user as defined by Table 3. The selection of the base, in this instance Medisca Transdermal Pain™ depends upon the desired viscosity of the topical cream. Within other embodiments of the invention a topical cream may include a topical foam.

TABLE 1
Baclofen Cream

| Ingredient | Quantity | Unit |
|---|---|---|
| Baclofen | 3.0 | g |
| Cyclobenzaprine hydrochloride | 2.0 | g |
| (a muscle relaxer) | | |
| Diclofenac sodium (a NSAID) | 5.0 | g |
| Lidocaine hydrochloride | 5.0 | g |
| (a sodium channel blocker) | | |
| DMSO (a solvent) | 7.5 | g |
| Transdermal Pain Base | 76.67 | g |
| Liquid Gel Complex | As Required | |

It would be evident to one of skill in the art that the quantities listed in Table 3 are representative of a preparation as may be made by a pharmacist or other individual. It would be evident that these quantities may be scaled to smaller and/or larger quantities according to the production requirements. Further, the quantities listed are representative and may be varied according to the specific percentage of topical agent required and/or by varying one or more of the non-pharmaceutical components within predetermined ranges and/or their elimination. For example, the diclofenac sodium may be 5.0±5.0 g or alternatively the DMSO may be 7.5±5.0 g as may the transdermal pain base.

Process 4: Powder to Liquid Preparation

An exemplary process for the preparation of a homogenous liquid-like dispersion of the tofacitinib citrate according to an embodiment of the invention comprises the following steps:
Step 4A: Combine and triturate the following ingredients together to form tofacitinib citrate to form a fine, homogenous powder.
   Micronized baclofen
   Micronized cyclobenzaprine hydrochloride
   Micronized diclofenac sodium
   Micronized ketamine hydrochloride
   Lidocaine hydrochloride
   An example of a micronization process is to spin at 1,000 rpm for 30 seconds-60 seconds using zirconium beads and then sieve.
Step 4B: Levigate the fine homogeneous powder blend from step 4A with 3 ml of propylene glycol.
Step 4C: Spin using a Mazerustar Revolutionary Planetary Mixer at 1,000-2,000 rpm for 30 seconds-60 seconds depending upon the total weight.

Process 5: Powder-Liquid to Medium Incorporation

An exemplary process for the preparation of the medium from the homogenous liquid-like dispersion to the base according to an embodiment of the invention comprises the following steps:
Step 5A: Incrementally add the homogeneous liquid-like dispersion from step 4C to the Transdermal Pain Base whilst continuously mixing, using a high-shear mixing technique (for example using a Mazerustar Revolutionary Planetary Mixer at 2000 rpm for 2 minutes)
Step 5B: The resultant homogenous cream-like dispersion where the cream/gel is de-aired and non-gritty.

Process 6: Viscosity Adjustment

An exemplary process for adjusting the final product viscosity according to an embodiment of the invention comprises the following steps:
Step 6A: If the final result is not thick enough, incrementally add LiquidGel Complex™, about 0.5 mL at a time, to the homogenous cream-like dispersion from Process 5 and thoroughly mix for 2 minutes at 2,000 rpm. Repeat the procedure until the desired viscosity is attained.
Typically the LiquidGel Complex™ should be within the range 1% to 6%.
If the final result is gritty then pass it through an ointment mill until it becomes smooth and uniform. Alternatively, discard the batch and begin again.

Process 7: Product Transfer

An exemplary process for the final stage of forming a topical cream according to an embodiment of the invention comprises the following steps:
Step 7A: Transfer final product from Process 6 to designated dispensing container(s).
If a transdermal foam base is employed within rather than a cream base then a foam dispensing pump should be employed for dispensing.

Process 8: Product Labelling

Once the cream-based (or foam-based) tofacitinib citrate is packaged in the dispensing container(s) then the dispensing container(s) should be labelled in compliance with the product labelling, drug regulations, etc. in the jurisdiction that the topical finasteride foam will be sold. For example, the product labelling may include for example the following elements as per United States pharmacopeia (USP Chapter 795).
Packaging Tightly closed, light-resistant ointment tube/jar/etc. Administer with metered dose-measuring device.
Estimated Beyond-Use 31 days.
Label 1 Use as prescribed. Do not exceed prescribed dose.
Label 2 Keep out of the reach of children.
Label 3 Cap tightly after use.
Label 4 For external use only.
Label 5 Keep in a dry place.
Label 6 Controlled substance. Dangerous unless used as directed.
Label 7 May produce psychological and/or physical dependence.
Label 8 May impair mental and/or physical ability. Use care when operating a car or machinery.
Label 9 Do not take with alcohol, sleep aids, tranquilizers or other CNS depressants.
Label 10 Do not apply to open wounds, areas of the skin that are damaged or blistered, deep wounds or large areas.
Storage Label 1 Room temperature storage (20° C.-23° C.).
Storage Label 2 Protect from light.
Storage Label 3 Shake well before use.

Add auxiliary labels specific to the API to the dispensing container as deemed necessary including but not limited to:
Add auxiliary labels specific to the API to the dispensing container as deemed necessary including but not limited to:
Contact the pharmacist in the event of adverse reactions.
Consult health care practitioner if any prescription or over-the-counter medications are currently being used or are prescribed for future use.
The quantity of the API administered is directly dependent on the quantity of the product applied.

Optionally, the dosage of the baclofen may be varied within the range 1.5 g to 6.0 g for the same mass of cream and other pharmaceutical ingredients. Within alternative formulations as noted supra the weights employed of the other ingredients may be individually varied or varied in combination.

C: Variants

Whilst the exemplary embodiment of the invention described supra exploits a topical cream as part of the pharmaceutical composition in combination with tofacitinib or baclofen it would be evident to one of skill in the art that such cream-based compositions may comprise one or more pharmaceutical ingredients providing a janus kinase inhibitor (in the case of tofacitinib) or GABA receptor activator (in the case of baclofen) together with one or more pharmaceutically acceptable excipients as required to prepare a dosage form for the effective delivery of the active agent. Accordingly, a topical finasteride composition may exploit a foam. Accordingly, alternate pharmaceutical compositions according to embodiments of the invention may exploit solutions, ointments, creams, gels, lotions, suspensions, and sprays as well as foams. Delivery of the janus kinase inhibitor (tofacitinib) or GABA receptor activator (baclofen) may exploit microspheres, microemulsions, nanoemulsions, nanoparticles, nanosuspensions, dermal sticks, roll-ons, pumps, patches, tapes, and the like.

Accordingly, a topical tofacitinib or baclofen composition such as the cream or foam means a composition that is applied onto the skin surface. Such a topical tofacitinib or baclofen composition may act “locally” or “transdermally”. A transdermal composition refers to a composition that can be applied to the body surface, which then may permeate through the stratum corneum or scalp to form a reservoir just beneath the skin surface or may be absorbed systemically to provide desirable drug levels in the circulation. Such a reservoir being a “skin depot” or “depot” for the pharmaceutical composition that provides storage of drug within the skin and releases the contained drug to surrounding tissue over a prolonged period of time, and/or delayed to commence after a period of time.

Within embodiments of the invention the topical formulation may include a penetration enhancement or permeation enhancement as a means to increase the permeability of a biological membrane (i.e. scalp) to a drug, e.g. tofacitinib or baclofen, so as to increase the rate at which the tofacitinib or baclofen is transported through the membrane.

Within embodiments of the invention suitable carriers for the topical tofacitinib or baclofen composition may include, but are not limited to, paraffin oils; esters of C8-C18 organic acids such as isopropyl myristate for example; C8-C30 fatty alcohols; silicone oils; vegetable oils; fractionated or hydrogenated vegetable oils; monoglycerides; diglycerides; triglycerides; phospholipids; dimethyl isosorbide; volatile solvents; N-methylpyrrolidone; N,N-dimethylacetamide and N,N-dimethylformamide; dimethylsulphoxide; alcohols such as ethanol and isopropyl alcohol; glycols such as propylene glycol, polyethylene glycol and glycerol; cyclodextrins such as beta-cyclodextrin, beta-hydroxy cyclodextrin, gamma-cyclodextrin, and hydroxypropyl cyclodextrin; and any mixture or mixtures thereof.

Within embodiments of the invention other pharmaceutically suitable excipients include, but are not limited to, surfactants, co-surfactants, penetration enhancers, antioxidants, buffering agents, preservatives, viscosity modifying agents, chelating/complexing agents, coloring agents, perfumes, polymers, gelling agents, alcohols, liquid or semi-solid oily components, and any mixture or mixtures thereof.

These other excipients according to embodiments of the invention can serve more than one purpose, such as, for example, a surfactant or co-surfactant used in the present invention can also act as a penetration or permeation enhancer.

Pharmaceutical compositions according to embodiments of the invention may comprise at least one janus kinase inhibitor (tofacitinib) or GABA receptor activator (baclofen) as an active agent, one or more volatile solvents, one or more surfactants, optionally at least one penetration enhancer, and optionally one or more other pharmaceutically acceptable excipients.

According to embodiments of the invention the drug is dissolved or dispersed in a suitable pharmaceutically acceptable carrier. For example, when a composition of the present invention is in the form of a dispersion, it comprises at least one surfactant and optionally one or more co-surfactants along with a pharmaceutically acceptable carrier. A surfactant may be an anionic, cationic, non-ionic, or zwitterionic.

Suitable surfactants for use within a topical composition according to embodiments of the invention may include, but not be limited to, sodium laurate, sodium stearate, sodium lauryl sulfate, cetyl trimethyl ammonium bromide, benzalkonium chloride, a poloxamer (for example 231, 182, and 184 where the first digit (two digits in a three-digit number) in the numerical designation, multiplied by 300, indicates the approximate molecular weight of the hydrophobe; and the last digit x 10 gives the percentage polyoxyethylene content), a polyoxyethylene sorbitan ester, lecithin, and any mixture or mixtures thereof.

Other suitable surfactants for use within a topical composition according to embodiments of the invention may include, but not be limited to, glycerol fatty acid esters such as glycerol monostearate, glycol fatty acid esters such as propylene glycol monostearate, polyhydric alcohol fatty acid esters such as polyethylene glycol monooleate, polyoxyethylene fatty acid esters such as polyoxyethylene stearate, polyoxyethylene fatty alcohol ethers such as polyoxyethylene stearyl ether, polyoxyethylene sorbitan fatty acid esters such as polyoxyethylene sorbitan monostearate, sorbitan esters such as sorbitan monostearate, alkyl glycosides such as cetearyl glucoside, sulfated oils such as a sulfuric ester of ricinoleic acid disodium salt, and sulfonated compounds such as alkyl sulfonates including sodium cetane sulfonate, amide sulfonates such as sodium N-methyl-N-oleyl laurate, sulfonated dibasic acid esters such as sodium dioctyl sulfosuccinate, alkyl aryl sulfonates such as sodium dodecylbenzene sulfonate, alkyl naphthalene sulfonates such a sodium isopropyl naphthalene sulfonate, a petroleum sulfonate such as aryl naphthalene with alkyl substitutes. Examples of suitable cationic surfactants include amine salts such as octadecyl ammonium chloride.

Suitable penetration enhancers for use within a topical composition according to embodiments of the invention may include, but not be limited to, sulfoxides such as dimethylsulfoxide (DMSO) and decylmethylsulfoxide (C10 MSO); ethers such as diethylene glycol monoethyl ether (available commercially as Transcutol™) and diethylene glycol monomethyl ether; 1-substituted azacycloheptan-2-ones, such as 1-n-dodecyl-cyclazacycloheptan-2-one; alcohols such as propanol, octanol, benzyl alcohol, and the like; fatty acids such as lauric acid, oleic acid, and valeric acid; fatty acid esters such as isopropyl myristate, isopropylpalmitate, methylpropionate, and ethyl oleate; polyol esters such as butanediol and polyethylene glycol monolaurate, amides and other nitrogenous compounds such as urea, N,N-dimethylacetamide (DMA), N,N-dimethylformamide (DMF), 2-pyrrolidone, 1-methyl-2-pyrrolidone, ethanolamine, diethanolamine, and triethanolamine; terpenes and terpinoids; alkanones; organic acids, such as salicylic acid and salicylates, citric acid and succinic-acid and the like; and any mixture or mixtures thereof.

Antioxidants for use within a topical composition according to embodiments of the invention may include, but not be limited to, for use within a topical composition according to embodiments of the invention may include, but not be limited to, tocopherol succinate, ascorbic acid, propyl gallate, vitamin E, butylated hydroxytoluene, butylated hydroxyanisole, sodium pyrosulfite, kojic acid, cysteine, hydroquinone, and the like, including any mixture or mixtures thereof.

Buffering agents for use within a topical composition according to embodiments of the invention may include, but not be limited to, alkali metal salts such as potassium and sodium carbonates, acetates, borates, phosphates, citrates and hydroxides; weak acids such as acetic, boric and phosphoric acids, and the like; and mixture or mixtures thereof.

Preservatives for use within a topical composition according to embodiments of the invention may include, but not be limited to, diazolidinyl urea and imidazolidinyl urea, as well as methyl, ethyl, propyl and butyl esters of p-hydroxybenzoic acid (parabens), isothiazolones, and the like, including any mixtures thereof.

Viscosity modifying agents for use within a topical composition according to embodiments of the invention may include, but not be limited to, cetyl alcohol, glycerol, polyethylene glycol (PEG), PEG-stearate, xanthan gums such as Keltrol™, and the like, including any mixture or mixtures thereof.

Chelating or complexing agents for use within a topical composition according to embodiments of the invention may include, but not be limited to, ethylenediaminetetraacetic acid (EDTA) and its derivatives, thioglycolic acid, thiolactic acid, thioglycerol, and the like, including mixture or mixtures thereof.

Polymers for use within a topical composition according to embodiments of the invention may include, but not be limited to, bioadhesive agents, gelling agents, film-forming agents, phase change agents, and any mixtures thereof. Examples of polymers include, but are not limited to, ethylcelluloses, acrylates, methacrylates, pyrrolidone polymers, including polymers of N-vinylpyrrolidone, polyoxyethylenes, hydroxypropyl methylcelluloses, hydroxypropyl celluloses, polymethylmethacrylates, cellulose acetates and their derivatives, cellulose acetate phthalates, hydroxypropyl methylcellulose phthalates, shellac, methacrylic acid based polymers such as those sold under the trademark EUDRAGIT™, zein, polycarbonates, polyorthoesters, polydioxanones, polyacetals, polyhydroxybutyrates, polyhydroxy valerates, polyethers, polyphosphazenes, polyhydroxy celluloses, polyalkylene oxalates, polyorthocarbonates, polyphosphoesters, star-branched polymers and copolymers, polysaccharides, polyketals, polyalkylene succinates, polypropylene oxides, chitin, chitosan, and other polymers known to a person skilled in the art of drug delivery, including copolymers, terpolymers, combinations and the likes, and any mixtures thereof.

Gelling agents for use within a topical composition according to embodiments of the invention may include, but not be limited to, cellulose and its derivatives, such as sodium carboxymethyl celluloses and hydroxyalkyl and alkyl celluloses, carbomers such as Carbopol™ and their derivatives, carob, carregeenans and derivatives, xanthan gum, sclerane gum, long chain alkanolamides, bentone and derivatives, kaolin, green clay, bentonite, magnesium aluminum silicate (Veegum™), guar gums (such as Jaguar™ HP-120), cross-linked acrylic acid polymers, and the like, including any mixtures thereof.

Liquid oily components for use within a topical composition according to embodiments of the invention may include, but not be limited to, sunflower oil, soybean oil, peanut oil, canola oil, cottonseed oil, coconut oil, palm oil, palm kernel oil, corn oil, flax seed oil, olive oil, safflower oil, fish oil, liquid state triglyceride esters of fatty acids, and the like, including any mixtures thereof.

Semi-solid or solid oily components for use within a topical composition according to embodiments of the invention may include, but not be limited to, C12-C30 higher fatty acids, e.g., stearic acid and linoleic acid, solid state mono-, di- and tri-glyceride esters of fatty acids, higher saturated alcohols, including aliphatic alcohols having 14-30 carbon atoms such as cetostearyl alcohol, waxes, such as carnauba wax, hydrocarbons, such as soft and hard paraffins, sphingolipids, and the like, including any mixtures thereof.

In embodiments of the present invention, pharmaceutical compositions provide topical delivery of janus kinase inhibitor (tofacitinib) or GABA receptor activator (baclofen) to enhance the availability of the active agent to the transdermal regions of different regions of the patient's (user's) body. In embodiments, pharmaceutical compositions of the present invention, upon administration, permit the drug to penetrate through the skin or the scalp and thereby provide pharmacologically effective systemic drug levels. In embodiments, compositions of the present invention form a depot or a reservoir at or near the point of application and exhibit a pharmacological effect for an extended duration of time, and/or commencing after a delayed time, after application. Further, compositions of the present invention are easy to formulate and frequently are removable by water washing. Also, in aspects, the compositions of the present invention have appreciable spreadability and can be easily applied to the skin.

Pharmaceutical compositions of the present invention comprising at least janus kinase inhibitor (tofacitinib) or GABA receptor activator (baclofen), can additionally comprise at least one another active agent. Such other active agents can include, but not be limited to, an ingredient to enhance or potentiate the activity of a janus kinase inhibitor (tofacitinib) or GABA receptor activator (baclofen) according to the topical cream formulation or are useful for management of any associated diseases/disorders, for which said janus kinase inhibitor (tofacitinib) or GABA receptor activator (baclofen) are indicated. In certain embodiments, such additional active agents may be chemical compounds or extracts of one or more active components obtained from a natural source, such as plant extracts.

Such additional active agents for a janus kinase inhibitor (tofacitinib) based topical cream or foam composition may include, but are not limited to: hair loss preventing agents; hair growth promoting agents; anti-alopecia agents such as alfatradiol, finasteride, dutasteride, epristeride, bexlosteride, izonsteride, lapisteride, turosteride, 17β-benzoyl-4-aza-5 alpha-androst-1-ene-3-one, 16-((4-chlorophenyl)oxy)-4,7-dimethyl-4-azaandronstan-3-one, 17β-carboxy-4-androsten-3-one, steroidal oximes and saw palmetto extract, FCE 28260, and minoxidil; anti-infectives; antibacterials; antifungals; antihistaminics; immunomodulatory agents; anti-dandruff agents; antivirals; antiandrogenic agents such as fluconazole, ketoconazole and spironolactone; hormones; steroids; and the like.

Such additional active agents for a GABA receptor activator (baclofen) based topical cream or foam composition may include, but are not limited to, anti-infectives; antibacterials; antifungals; antihistaminics; immunomodulatory agents; an anti-inflammatory agent; an analgesic such as capsacin, diclofenac sodium, lidocaine, methyl salicylate, and trolamine; a steroid such as triamcinolone, fluocinolone, betamethasone, desonide, diflorasone, clobetasol, desoximetasone, andhydrocortisone, and mometasone; antivirals, hormones, a muscle relaxant such as a neuromuscular blocker, a spasmolytic, cyclobenzaprine, metaxalone, orphenadrine (anticholinergic), chlorzoxazone, tizanidine (clonidine relative), diazepam, tetrazepam and other benzodiazepines, mephenoxalone, methocarbamol, and dantrolene; a sodium channel blocker such as lidocaine and lidocaine hydrochloride; and the like.

Optionally, a topical cream or foam according to an embodiment of the invention may include a perfume or a flavouring.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A pharmaceutical composition comprising:
a janus kinase inhibitor; and
a topical carrier for the janus kinase inhibitor.

2. The pharmaceutical composition according to claim 1, wherein
the janus kinase inhibitor is (3R,4R)-4methyl-3-(methyl-7H-pyrrolo [2,3-d]pyrimidin-4-ylamino)-ß-oxo-1-piperidinepropanenitrile, 2hydroxy-1,2,3-propanetricarboxylate (1:1) (tofacitinib).

3. The pharmaceutical composition according to claim 1, wherein
the topical carrier is a foam.

4. The pharmaceutical composition according to claim 1, wherein
the topical carrier is a cream.

5. The pharmaceutical composition according to claim 1, wherein
the janus kinase inhibitor comprises at least one of:
between a minimum of 1% of the pharmaceutical composition and a maximum of 5% of the pharmaceutical composition;
between a minimum of 1% of the pharmaceutical composition and a maximum of 3% of the pharmaceutical composition;
between a minimum of 1% of the pharmaceutical composition and a maximum of 2% of the pharmaceutical composition; and
between a minimum of 1.5% of the pharmaceutical composition and a maximum of 2.5% of the pharmaceutical composition.

6. The pharmaceutical composition according to claim 1, wherein
the janus kinase inhibitor is tofactinib citrate; and
the janus kinase inhibitor comprises at least one of:
between a minimum of 1% of the pharmaceutical composition and a maximum of 6% of the pharmaceutical composition;
between a minimum of 2% of the pharmaceutical composition and a maximum of 4% of the pharmaceutical composition; and
between a minimum of 2.5% of the pharmaceutical composition and a maximum of 3.5% of the pharmaceutical composition.

7. A pharmaceutical composition comprising:
a GABA receptor activator inhibitor; and
a topical carrier for the GABA receptor activator.

8. The pharmaceutical composition according to claim 7, wherein
the GAB A receptor activator is β-(4-chlorophenyl)-γ-aminobutyric acid (β-(4-chlorophenyl)-GAB A) (baclofen).

9. The pharmaceutical composition according to claim 7, wherein
the GABA receptor activator activates GABA_{B} receptors.

10. The pharmaceutical composition according to claim 7, wherein
the topical carrier is a foam.

11. The pharmaceutical composition according to claim 7, wherein
the topical carrier is a cream.

12. The pharmaceutical composition according to claim 7, wherein
the GABA receptor activator comprises at least one of:
between a minimum of 1% of the pharmaceutical composition and a maximum of 6% of the pharmaceutical composition;
between a minimum of 2% of the pharmaceutical composition and a maximum of 4% of the pharmaceutical composition; and
between a minimum of 2.5% of the pharmaceutical composition and a maximum of 3.5% of the pharmaceutical composition.

13. The pharmaceutical composition according to claim 7, wherein
the GABA receptor activator is baclofen; and
the GABA receptor activator comprises at least one of:
between a minimum of 1% of the pharmaceutical composition and a maximum of 6% of the pharmaceutical composition;
between a minimum of 2% of the pharmaceutical composition and a maximum of 4% of the pharmaceutical composition; and
between a minimum of 2.5% of the pharmaceutical composition and a maximum of 3.5% of the pharmaceutical composition.

14. The pharmaceutical composition according to claim 7, further comprising
a muscle relaxant;
an apolar aprotic solvent;
a sodium channel blocker; and
a non-steroidal anti-inflammatory drug.

15. The pharmaceutical composition according to claim 7, further comprising
a first predetermined percentage of diclofenac sodium;
a second predetermined percentage of lidocaine or lidocaine hydrochloride;
a third predetermined percentage of DMSO; and
a fourth predetermined percentage of cyclobenzaprine.
